(19) United States
(12) Patent Application Publication
(10) Pub. No.: US 2017/0020401 A1
(43) Pub. Date: Jan. 26, 2017

(54) BIOLOGICAL-INFORMATION DETECTING DEVICE

(71) Applicant: SEIKO EPSON CORPORATION, Tokyo (JP)

(72) Inventors: Tetsu NAKAYAMA, Matsumoto-shi (JP); Ryoichi NAKAGAWA, Matsumoto-shi (JP)

(73) Assignee: SEIKO EPSON CORPORATION, Tokyo (JP)

(21) Appl. No.: 15/217,915

(22) Filed: Jul. 22, 2016

(30) Foreign Application Priority Data
Jul. 24, 2015 (JP) 2015-146426

(51) Int. Cl. A61B 5/024; A61B 5/00

(52) U.S. Cl. CPC A61B 5/02438; A61B 5/681; A61B 5/02444

(57) ABSTRACT

A biological-information detecting device includes a case section worn on a subject, a detection window of a detector configured to detect biological information of the subject being provided on a first surface on the subject side of the case section, a band section attached to the case section, and a pad section provided around the detection window on the first surface of the case section. An air entry path for the air to enter the detection window is provided in the pad section.

### CROSS-REFERENCES TO RELATED APPLICATIONS

This application claims priority to Japanese Patent Application No. 2015-146426, filed Jul. 24, 2015, the entirety of which is herein incorporated by reference.

### BACKGROUND

1. Technical Field

The present invention relates to a biological-information detecting device and the like.

2. Related Art

In recent years, according to the improvement of a sensing technique, a large number of wristwatch-type activity meters (biological-information detecting devices) have been commercialized by various companies. Among the wristwatch-type biological-information detecting devices, there are biological-information detecting devices on which detectors (sensor sections) are provided on the rear sides of case sections, that is, subject sides. In particular, among such biological-information detecting devices, a biological-information detecting device including an optical sensor as the detector can more accurately measure a pulse wave compared with the biological-information detecting devices of the other types.

The optical sensor measures reflected light of light emitted from a light emitter to thereby measure a pulse wave. Therefore, when external light intrudes into the optical sensor, accuracy of pulse wave detection is deteriorated. As measures against this problem, there has been proposed a method of setting a doughnut-like bank around a detection window of the detector and increasing adhesion with a wrist to block the intrusion of light from the outside.

JP-A-2009-240511 (Patent Literature 1) discloses, as a technique for improving adhesion of a biometric device and a wrist, an invention for adopting a pad between the biometric device and the wrist.

When the bank is provided around the detection window of the detector, a small gap is formed between the wrist and the detection window. In this case, since the adhesion between the biological-information detecting device and an arm is improved, a space of the small gap is closed, air permeability is lost, and the air is not replaced. Therefore, during vigorous exercise such as running, steam and sweat remain as liquid in the space of the gap because of perspiration from the wrist. As a result, in some case, the detection window of the detector gets fogged, refraction and reflection of light due to moisture occur, and a pulse wave detection property is deteriorated. Since the sweat accumulated in the small gap moves because of vibration during exercise, discomfort is sometimes given to a wearer.

### SUMMARY

An advantage of some aspects of the invention is to provide a biological-information detecting device and the like that can suppress deterioration in pulse wave detection accuracy due to sweat and reduce discomfort of a wearer.

An aspect of the invention relates to a biological-information detecting device including: a case section worn on a subject, a detection window of a detector configured to detect biological information of the subject being provided on a first surface on the subject side of the case section; a band section attached to the case section; and a pad section provided around the detection window on the first surface of the case section. An air entry path for the air to enter the detection window is provided in the pad section.

In the aspect of the invention, the air entry path for the air to enter the detection window of the detector provided on the first surface of the case section is provided in the pad section. Therefore, it is possible to suppress deterioration in pulse wave detection accuracy due to sweat and reduce discomfort of a wearer.

In the biological-information detecting device according to the aspect of the invention, the pad section may include: a first pad section; and a second pad section, and the air entry path may include a first gap section provided between a first end portion of the first pad section and a first end portion of the second pad section opposed to the first end portion of the first pad section.

With this configuration, it is possible to, for example, replace the air in a gap between a wrist and the detection window via the first gap section.

In the biological-information detecting device according to the aspect of the invention, the air entry path may further include a second gap section provided between a second end portion of the first pad section and a second end portion of the second pad section opposed to the second end portion of the first pad section.

With this configuration, it is possible to, for example, replace the air in the gap between the wrist and the detection window via the second gap section.

In the biological-information detecting device according to the aspect of the invention, the first gap section may be a gap section extending along a longitudinal direction of the band section.

With this configuration, it is possible to suppress external light from being made incident on the detection window of the detector and suppress the deterioration in the pulse wave detection accuracy.

In the biological-information detecting device according to the aspect of the invention, the pad section may include first to third pad sections, and the air entry path may include: a first gap section provided between a first end portion of the first pad section and a first end portion of the second pad section opposed to the first end portion of the first pad section; and a second gap section provided between a second end portion of the second pad section and a first end portion of the third pad section opposed to the second end portion of the second pad section.

With this configuration, it is possible to, for example, cause the air to flow into the gap between the wrist and the detection window from at least two directions.

In the biological-information detecting device according to the aspect of the invention, the pad section may further include a fourth pad section, and the air entry path may include a third gap section provided between a second end portion of the third pad section and a first end portion of the fourth pad section opposed to the second end portion of the third pad section.

With this configuration, it is possible to, for example, cause the air to flow into the gap between the wrist and the detection window from at least three directions.

In the biological-information detecting device according to the aspect of the invention, the air entry path may include a fourth gap section provided between a second end portion of the fourth pad section and a second end portion of the first pad section opposed to the second end portion of the fourth pad section.

With this configuration, it is possible to, for example, cause the air to flow into the gap between the wrist and the detection window from at least four directions.

In the biological-information detecting device according to the aspect of the invention, the air entry path may be an entry path having a bent shape or a curved shape.

With this configuration, compared with when the air entry path is linear, it is possible to, for example, further suppress intrusion of external light into the gap between the wrist and the detection window.

In the biological-information detecting device according to the aspect of the invention, the pad section may include: a first pad section provided around the detection window; and a second pad section provided around the first pad section, in the first pad section, a first gap section may be provided on a first direction side of the detection window, and, in the second pad section, when a direction crossing the first direction is represented as a second direction, a second gap section may be provided on the second direction side of the detection window.

With this configuration, it is possible to, for example, complicatedly bend an air passage and suppress intrusion of external light into the gap between the wrist and the detection window.

In the biological-information detecting device according to the aspect of the invention, the pad section may include: a first pad section; and a second pad section, the air entry path may include a first gap section, and, when an opposite direction of the first direction is represented as a second direction and a direction crossing the first direction is represented as a third direction, a first gap section end portion of the first gap section may be located on the first direction side of a first end of the first pad section and a second gap section end portion of the first gap section may be located on the second direction side of a first end portion of the second pad section and on the third direction side of the first gap section end portion.

With this configuration, it is possible to bend the air entry path to block intrusion of external light. In addition, since simple shapes are combined, it is possible to, for example, iconize a shape and obtain a shape with a high design property.

In the biological-information detecting device according to the aspect of the invention, the pad section may include: a first pad section; and a second pad section, and the second pad section may be a pad section point-symmetrical to the first pad section centering on the detection window.

With this configuration, it is possible to bend the air entry path to block intrusion of external light. In addition, since simple shapes are combined, it is possible to, for example, iconize a shape and obtain a shape with a high design property.

In the biological-information detecting device according to the aspect of the invention, the air entry path may include: a first gap section; and a second gap section, and the second gap section may be a gap section point-symmetrical to the first gap section centering on the detection window.

With this configuration, it is possible to bend the air entry path to block intrusion of external light. In addition, since simple shapes are combined, it is possible to, for example, iconize a shape and obtain a shape with a high design property.

In the biological-information detecting device according to the aspect of the invention, the pad section may be formed of a material softer than a material forming the first surface of the case section.

With this configuration, for example, since silicone gel has a heat radiation effect, it is possible to, for example, allow heat generated by perspiration to escape.

In the biological-information detecting device according to the aspect of the invention, the first surface of the case section may include a recessed section into which at least a part of the pad section is inserted.

With this configuration, it is possible to, for example, prevent the pad section from easily coming off the first surface.

### BRIEF DESCRIPTION OF THE DRAWINGS

The invention will be described with reference to the accompanying drawings, wherein like numbers reference like elements.

FIG. 1 is a perspective view of the upper surface (the front surface) of a biological-information detecting device in an embodiment.

FIG. 2 is a perspective view of the lower surface (the rear surface) of the biological-information detecting device in the embodiment.

FIG. 3 is an explanatory diagram of a worn state of the biological-information detecting device.

FIG. 4 is an explanatory diagram of a first surface of a case section and a pad section.

FIG. 5 is an explanatory diagram of a biological-information detecting device including two gap sections.

FIG. 6 is an explanatory diagram of a biological-information detecting device including one gap section.

FIG. 7 is an explanatory diagram of a biological-information detecting device including four gap sections.

FIG. 8 is an explanatory diagram of another biological-information detecting device including four gap sections.

FIG. 9 is an explanatory diagram of still another biological-information detecting device including four gap sections.

FIG. 10 is an explanatory diagram of a biological-information detecting device including point-symmetrical gap sections.

FIG. 11 is an explanatory diagram of another biological-information detecting device including point-symmetrical gap sections.

FIG. 12 is an explanatory diagram of still another biological-information detecting device including point-symmetrical gap sections.

FIG. 13 is an explanatory diagram of recessed sections provided on a first surface.

FIG. 14 is an explanatory diagram of a biological-information detecting device including one band.

### DESCRIPTION OF EXEMPLARY EMBODIMENTS

An embodiment is explained below. Note that the embodiment explained below does not unduly limit contents of the invention described in the appended claims. Not all of components explained in the embodiment are essential constituent elements of the invention.

### 1. Overview

Among wristwatch-type biological-information detecting devices, there are biological-information detecting devices on which detectors (sensor sections) are provided on the rear sides of case sections, that is, subject sides. In particular, among such biological-information detecting devices, a biological-information detecting device including an optical sensor as the detector can more accurately measure a pulse wave compared with the biological-information detecting devices of the other types. However, the optical sensor measures reflected light of light emitted from a light emitter to thereby measure a pulse wave. Therefore, when external light intrudes into a detection window, accuracy of pulse wave detection is deteriorated.

Therefore, in this embodiment, a doughnut-like bank is set around a detection window of a detector of a biological-information detecting device. For example, as the doughnut-like bank disposed around the detection window, a pad made of a soft material and the like are conceivable. Consequently, it is possible to improve adhesion with a wrist and, at the same time, block intrusion of light from the outside.

However, when the bank of the soft pad is provided around the detection window, whereas adhesion of the biological-information detecting device and an arm is improved, a small gap is formed between the wrist and the detection window. Since a space of the small gap is closed by the bank, air permeability is lost and the air is not replaced. Therefore, during vigorous exercise such as running, steam and sweat remain as liquid in the space of the gap because of perspiration from the wrist. As a result, in some case, the detection window of the detector gets fogged, refraction and reflection of light due to moisture occur, and a pulse wave detection property is deteriorated. In this way, whereas adhesion to the arm is improved by the use of the soft pad, sweat easily accumulates around the detection window. Accuracy of pulse wave detection is sometimes deteriorated. Since the sweat accumulated in the small gap moves because of vibration during exercise, discomfort is sometimes given to a wearer.

Therefore, in the biological-information detecting device in this embodiment, a pad section is provided to make it possible to suppress intrusion of external light into the detector. An air entry path is provided in the pad section to make it possible to replace the air around the detection window of the detector.

### 2. Configuration Example of the Biological-Information Detecting Device

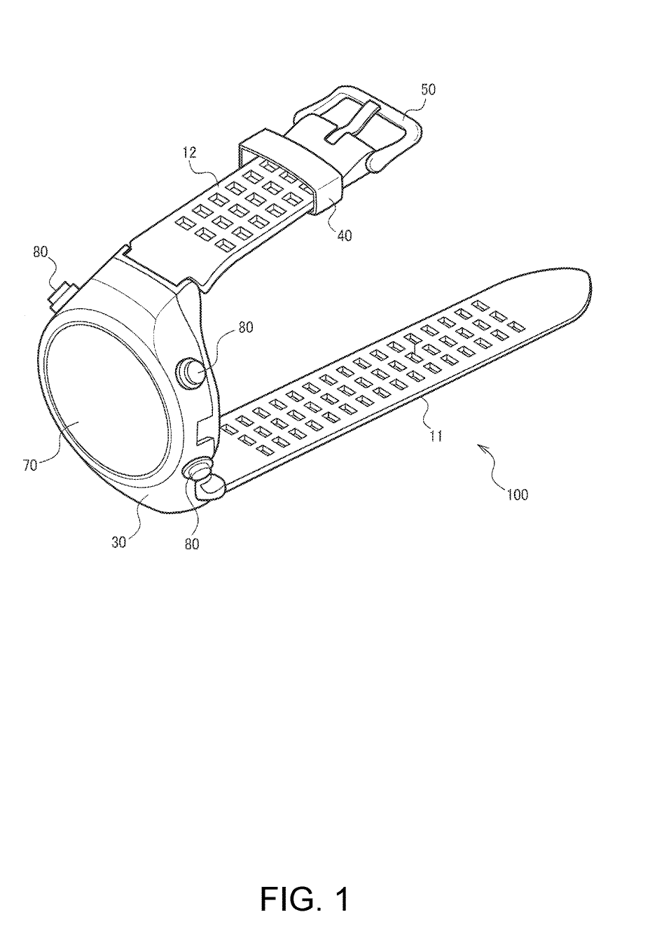

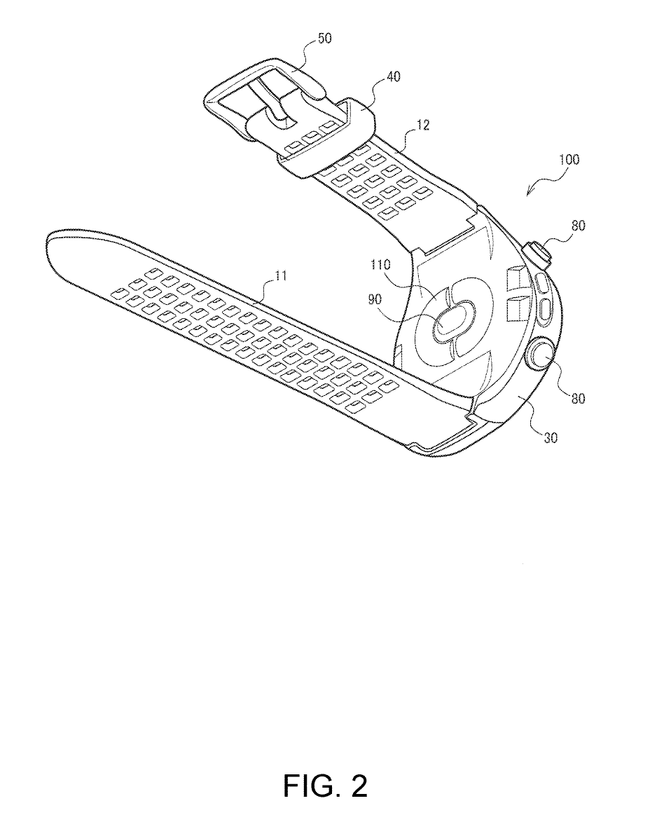

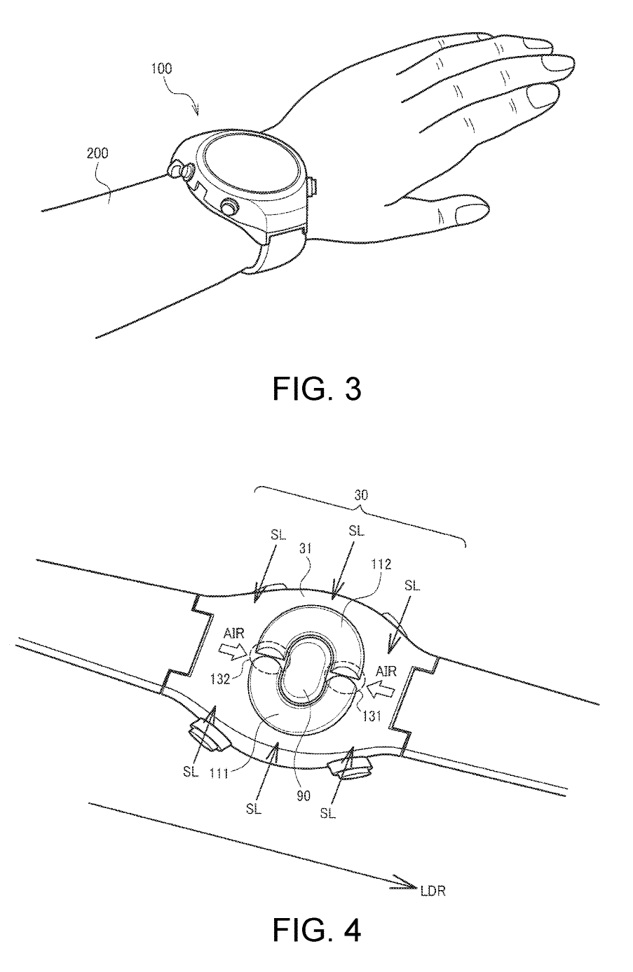

A configuration example of a biological-information detecting device 100 in this embodiment is shown in FIGS. 1 and 2. FIG. 1 is a perspective view of the upper surface (the front surface) of the biological-information detecting device 100. FIG. 2 is a perspective view of the lower surface (the rear surface) of the biological-information detecting device 100. A state in which the biological-information detecting device 100 shown in FIGS. 1 and 2 is worn on a wrist 200 of a subject is shown in FIG. 3. The upper surface (the front surface) of the biological-information detecting device 100 is a surface on a side where a display surface of a display 70 provided in a case section 30 explained below can be visually recognized. The lower surface (the rear surface) of the biological-information detecting device 100 is a surface on a side where a detection window 90 of a detector provided in the case section 30 explained below can be visually recognized. Alternatively, when a surface of the case section 30 in which the detector or the detection window 90 is provided is represented as the lower surface, a surface of the case section 30 most distant from the lower surface along the normal of the lower surface can also be referred to as upper surface. Alternatively, in a state in which the biological-information detecting device 100 is worn on the subject, a surface of the case section 30 in contact with the subject can also be referred to as lower surface. A surface of the case section 30 most distant from the lower surface along the normal direction of the lower surface can also be referred to as upper surface.

The biological-information detecting device 100 in this embodiment includes a first band section 11, a second band section 12, a case section 30, a fixer (a free ring or a fixed ring) 40, a buckle section (a coupler) 50, a display 70, operation sections 80, a detector (a sensor section), and a pad section 110. However, the biological-information detecting device 100 is not limited to the configuration shown in FIGS. 1 and 2. Various modified implementations such as omission of a part of the components and addition of other components are possible.

The sections of the biological-information detecting device 100 are explained. The first band section 11 is attached to one end of the case section 30. The second band section 12 is attached to the other end of the case section 30. The case section 30 includes a first surface 31 that faces a subject side when the biological-information detecting device 100 is worn on the subject.

In the case section 30, the detection window 90 of the detector (the sensor section), which detects biological information of the subject, is provided on the first surface 31. As shown in FIG. 4, the first surface 31 is a part of the surface of the case section 30, which is on the subject side when the biological-information detecting device 100 is worn.

The detector includes a not-shown light emitter, a not-shown light receiver, and the detection window 90. The detector detects the biological information of the subject in a state in which the detector is pressed against the subject. Specifically, light emitted from the light emitter is radiated on the subject via the detection window 90. The light reflected from the subject is made incident on the light receiver again via the detection window 90. The detector detects the biological information of the subject on the basis of the reflected light received by the light receiver. The detection window 90 is formed of a transparent member or a semitransparent member that transmits the light emitted from the light emitter and the reflected light reflected from the subject. More specifically, for example, the detection window 90 is formed of transparent plastic or the like. During the wearing, the detection window 90 plays a role of physically separating the light emitter and the light receiver from the subject. The light receiver can be realized by a light receiving element such as a photodiode. The light emitter can be realized by a light emitting element such as an LED.

The buckle section (the coupler) 50 couples the first band section 11 and the second band section 12. The fixer (the free ring or the fixed ring) 40 fixes the first band section 11 along the second band section 12 in a state in which the first band section 11 and the second band section 12 are coupled. Further, the display 70 displays various kinds of information. The operation sections 80 receive operation by a user.

The pad section 110 is provided around the detection window 90 of the first surface 31 of the case section 30. An air entry path for the air to enter the detection window 90 is provided in the pad section 110. The provision of the pad section 110 around the detection window 90 means that the pad section 110 is provided within a given distance range centering on the detection window 90. In this case, the pad section 110 does not always need to be provided in the entire range of the detection window 90. That is, for example, the pad section 110 may be provided only on a given direction side with the detection window 90 set as a reference position. Consequently, as shown in FIG. 4, it is possible to replace the air in a gap between the wrist 200 of the subject and the detection window 90.

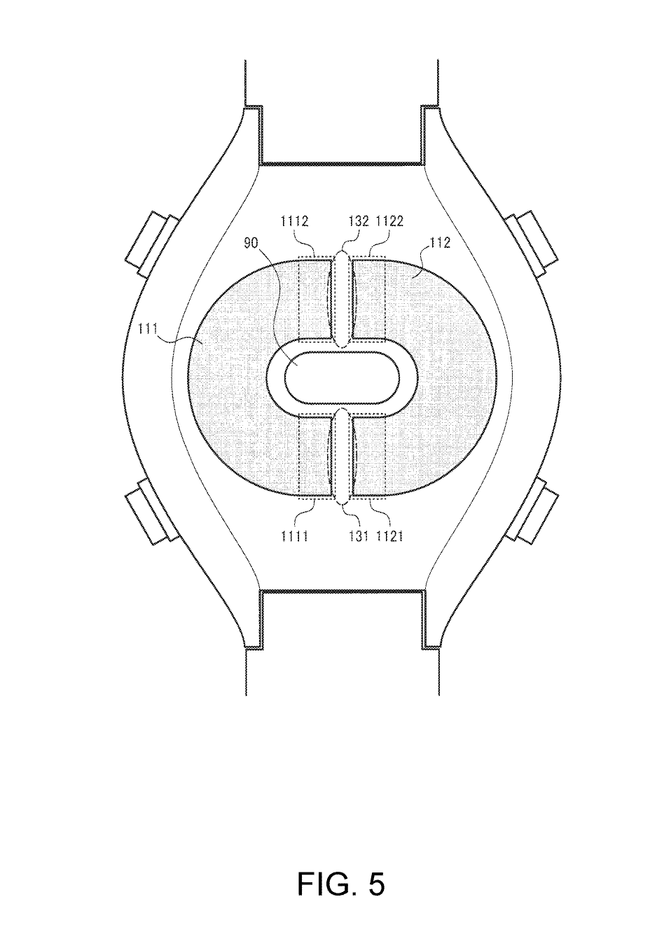

Specifically, in FIG. 4, the first surface 31 side of the case section 30 is shown. In an example shown in FIG. 4, as explained in detail below with reference to FIG. 5, the pad section 110 includes a first pad section 111 and a second pad section 112. Gap portions between the first pad section 111 and the second pad section 112 form an air entry path (131 and 132) for allowing the air to pass to the gap between the wrist 200 and the detection window 90.

In this case, external light SL is blocked by the pad sections 111 (112). Therefore, it is possible to prevent the external light SL from being made incident on the detection window 90 of the detector. Since air AIR flows into the gap between the detection window 90 and the wrist 200 through the air entry path (131 and 132), it is possible to circulate the air in the gap between the wrist 200 and the detection window 90. As a result, it is possible to prevent the detection window 90 from getting fogged, prevent refraction and reflection of light due to moisture from occurring, and suppress deterioration in accuracy of pulse wave detection. It is also possible to prevent sweat from accumulating in the gap between the wrist 200 and the detection window 90. Therefore, it is also possible to prevent discomfort due to sweat from being given to the wearer. As explained above, according to this embodiment, it is possible to suppress deterioration in pulse wave detection accuracy due to sweat during exercise and reduce discomfort of the wearer.

The pad section 110 is explained in detail with reference to FIGS. 5 to 12. In FIGS. 5 to 12, the pad section 110 provided on the first surface 31 (the rear surface) of the case 30 is shown.

First, in an example shown in FIG. 5, the pad section 110 includes the first pad section 111 and the second pad section 112. The air entry path includes a first gap section 131. The first gap section 131 is provided between a first end portion 1111 of the first pad section 111 and a first end portion 1121 of the second pad section 112 opposed to the first end portion 1111 of the first pad section 111.

The first end portion 1111 of the first pad section 111 indicates a predetermined range at one end of the first pad section 111 as indicated by a dotted line in FIG. 5. Similarly, the first end portion 1121 of the second pad section 112 indicates a predetermined range at one end of the second pad section 112 opposed to the first end portion 1111 of the first pad section 111.

Consequently, it is possible to, for example, replace the air in the gap between the wrist 200 and the detection window 90 via the first gap section 131.

As shown in FIG. 5, the air entry path further includes a second gap section 132. The second gap section 132 is provided between a second end portion 1112 of the first pad section 111 and a second end portion 1122 of the second pad section 112 opposed to the second end portion 1112 of the first pad section 111.

The second end portion 1112 of the first pad section 111 indicates a predetermined range at the other end different from the first end portion 1111 of the first pad section 111 as indicated by a dotted line in FIG. 5. Similarly, the second end portion 1122 of the second pad section 112 is opposed to the second end portion 1112 of the first pad section 111 and indicates a predetermined range at the other end different from the first end portion 1121 of the second pad section 112.

Consequently, it is possible to, for example, replace the air in the gap between the wrist 200 and the detection window 90 via the second gap section 132. Since not only the first gap section 131 but also the second gap section 132 is present, it is possible to allow the air to more easily pass through the gap between the wrist 200 and the detection window 90. Note that, in this example, since a wearing part of the biological-information detecting device 100 is the wrist 200 of the subject, it is described that it is possible to replace the air in the gap between the wrist 200 and the detection window 90. However, in this embodiment, the wearing part of the biological-information detecting device 100 is not limited to the wrist 200 of the subject. That is, in this embodiment, it is possible to replace the air in gaps between, besides the wrist 200, wearing parts such as an arm, a foot, an abdomen, and the like and the detection window 90.

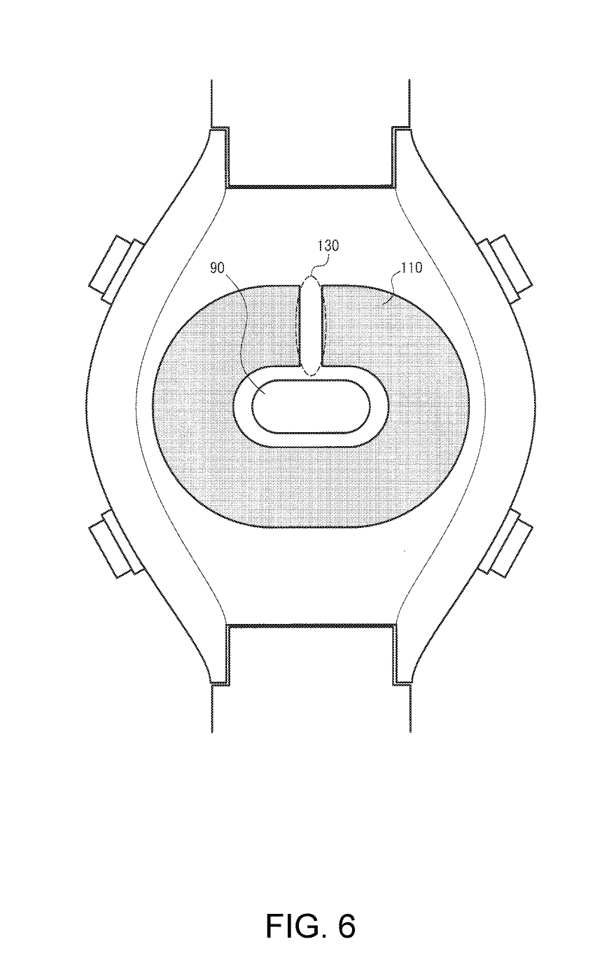

However, in this embodiment, the air entry path does not always include a plurality of gap sections. As shown in FIG. 6, the air entry path may include only one gap section 130.

The gap section means a gap that connects the gap between the detection window 90 and the wrist 200 and a space on the outside. The gap between the detection window 90 and the wrist 200 strictly means a gap surrounded by the first surface 31 of the case section 30, the detection window 90 of the detector, the pad section 110, and the wrist 200. However, as explained above, in this embodiment, the wearing part of the biological-information detecting device 100 is not limited to the wrist 200. Therefore, the gap section can also be considered a gap that connects a gap surrounded by the first surface 31 of the case section 30, the detection window 90 of the detector, the pad section 110, and the wearing part to the space on the outside. Note that, as shown in FIGS. 5 and 6, the gap section may be a slit of the pad section 110 or may be a hole opened in the pad section 110. Further, expressed in another way, the pad section 110 can also be considered as having a discontinuous region.

The air entry path includes other gaps or holes (openings) for the air to enter the gap surrounded by the first surface 31 of the case section 30, the detection window 90 of the detector, the pad section 110, and the wearing part.

As shown in FIG. 4 referred to above, the first gap section 131 may be a gap section extending along a long side direction LDR of the band section 11 (12). In other words, when the detection window 90 of the detector is set as a reference point, the gap section may be provided in a direction parallel to the long side direction LDR of the band section 11 (12). That is, it is possible to secure an air passage in a direction orthogonal to (crossing) a direction in which external light intrudes into the detection window 90 of the detector when the biological-information detecting device 100 is worn on the wrist 200.

Consequently, it is possible to suppress the external light from being made incident on the detection window 90 of the detector and suppress deterioration in pulse wave detection accuracy.

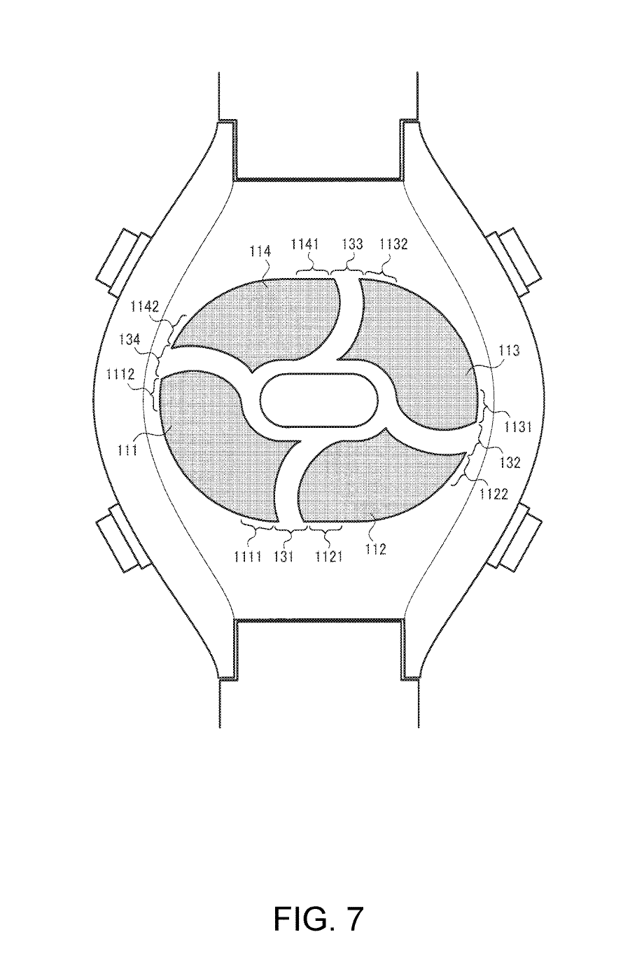

As shown in FIG. 7, the pad section 110 may include two or more pad sections. For example, as shown in FIG. 7, the pad section 110 may include first to third pad sections 111 to 113. In this case, the air entry path includes the first gap section 131 and the second gap section 132.

Specifically, the first gap section 131 is provided between the first end portion 1111 of the first pad section 111 and the first end portion 1121 of the second pad section 112 opposed to the first end portion 1111 of the first pad section 111. The second gap section 132 is provided between the second end portion 1122 of the second pad section 112 and a first end portion 1131 of the third pad section 113 opposed to the second end portion 1122 of the second pad section 112.

Consequently, it is possible to, for example, cause the air to flow into the gap between the wrist 200 and the detection window 90.

As shown in FIG. 7, the pad section 110 may further include a fourth pad section 114. In this case, the air entry path further includes a third gap section 133. Specifically, the third gap section 133 is provided between a second end portion 1132 of the third pad section 113 and a first end portion 1141 of the fourth pad section 114 opposed to the second end portion 1132 of the third pad section 113.

That is, in an example shown in FIG. 7, the air entry path may include a third gap section (the third gap section 133) in addition to the two gap sections (the first gap section 131 and the second gap section 132). More specifically, in the example shown in FIG. 7, the first to fourth pad sections 111 to 114 are circularly disposed adjacent to one another in order to surround the detection window 90. That is, the first pad section 111 is disposed to be adjacent to the second pad section 112 and the fourth pad section 114. The second pad section 112 is disposed to be adjacent to the first pad section 111 and the third pad section 113. The third pad section 113 is disposed to be adjacent to the second pad section 112 and the fourth pad section 114. In this case, the first pad section 111 and the third pad section 113 are disposed in positions opposed to each other across the detection window 90. The second pad section 112 and the fourth pad section 114 are disposed in positions opposed to each other across the detection window 90.

A gap between the first pad section 111 and the second pad section 112 forms the first gap section 131. A gap between the second pad section 112 and the third pad section 113 forms the second gap section 132. A gap between the third pad section 113 and the fourth pad section 114 forms the third gap section 133.

Consequently, it is possible to, for example, cause the air to flow into the gap between the wrist 200 and the detection window 90 from at least three directions.

Further, as shown in FIG. 7, the air entry path may include a fourth gap section 134. Specifically, the fourth gap section 134 is provided between a second end portion 1142 of the fourth pad section 114 and the second end portion 1112 of the first pad section 111 opposed to the second end portion 1142 of the fourth pad section 114. That is, as explained above, when the four pad sections are circularly disposed centering on the detection window 90, a gap between the fourth pad section 114 and the first pad section 111 forms the fourth gap section 134.

Consequently, it is possible to, for example, cause the air to flow into the gap between the wrist 200 and the detection window 90 from at least four directions. Since the number of gap sections increases, it is possible to, for example, allow the air to more easily flow into the gap between the wrist 200 and the detection window 90.

In the example shown in FIG. 7, the air entry path is an entry path having a curved shape. In other words, the first to fourth gaps sections 131 to 134 form the entry path having the curved shape.

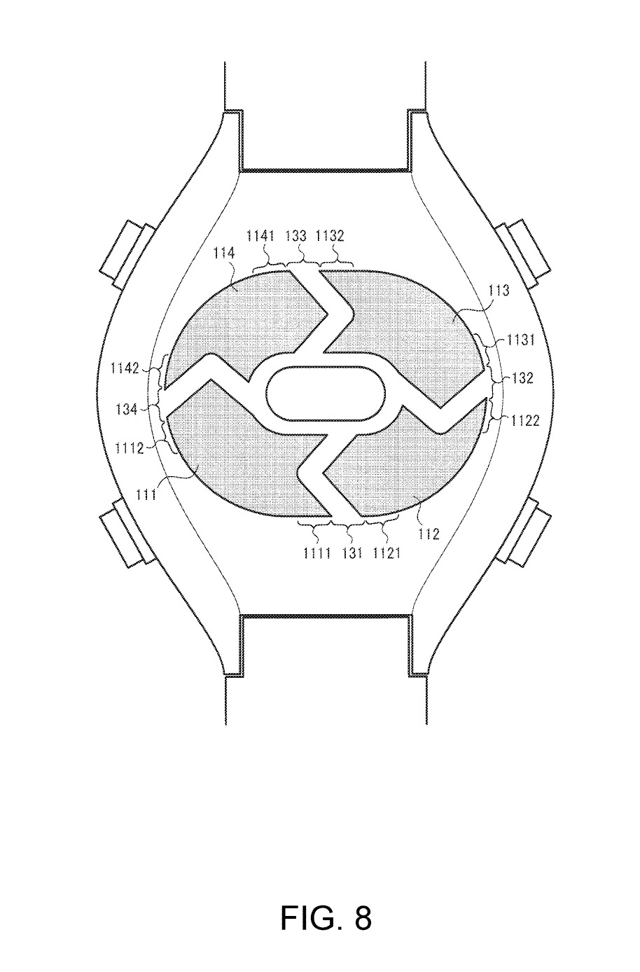

Further, as a modification, the air entry path (the first to fourth gap sections 131 to 134) may be an entry path having a bent shape shown in FIG. 8.

Consequently, compared with when the air entry path is linear, it is possible to, for example, further suppress intrusion of external light into the gap between the wrist 200 and the detection window 90.

The first gap section 131 and the third gap section 133 disposed along the long side direction LDR of the band section 11 (12) shown in FIG. 4 may be formed in a linear shape as shown in FIG. 5 or 6. The second gap section 132 and the fourth gap section 134 disposed along a direction orthogonal to the long side direction LDR may be formed in a curved shape and a bent shape as shown in FIGS. 7 and 8. With such a configuration, it is possible to provide air permeability while securing a light blocking property. Besides, various combinations are possible concerning the shape of the gap sections.

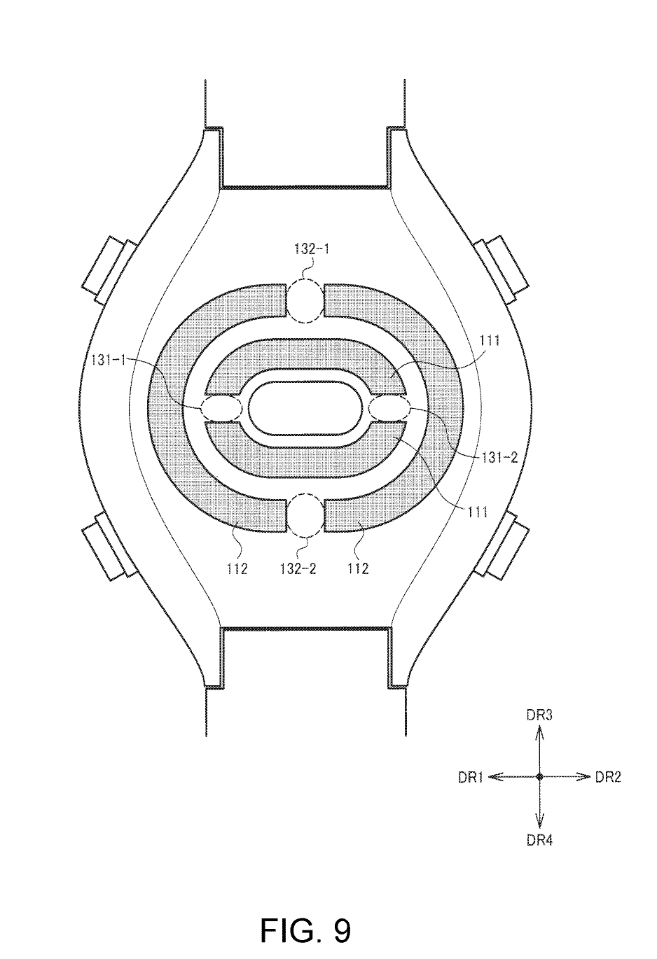

The pad section 110 may have a double doughnut shape shown in FIG. 9. In the case of an example shown in FIG. 9, the pad section 110 includes the first pad section 111 provided around the detection window 90 and the second pad section 112 provided around the first pad section 111. Each of the first pad section 111 and the second pad section 112 is configured by two pads.

In the following explanation, the direction orthogonal to the long side direction LDR of the band section 11 (12) is represented as a first direction DR1 and the opposite direction of the first direction DR1 is represented as a second direction DR2. A direction along the long side direction LDR of the band section 11 (12) and orthogonal to the first direction DR1 and the second direction DR2 is represented as a third direction DR3 and the opposite direction of the third direction DR3 is represented as a fourth direction DR4.

In the first pad section 111, a first gap section 131-1 (or 131-2) is provided on the first direction DR1 (or the second direction DR2) side of the detection window 90. In the second pad section 112, when a direction crossing (orthogonal to) the first direction DR1 (or the second direction DR2) is represented as the third direction DR3 (or the fourth direction DR4), a second gap section 132-1 (or 132-2) is provided on the third reaction DR3 (or the fourth direction DR4) side of the detection window 90.

Consequently, it is possible to, for example, complicatedly bend an air passage and suppress intrusion of external light into the gap between the wrist 200 and the detection window 90. Compared with the pad shape shown in FIGS. 5 to 8, since the capacity of the pad is small, it is also possible to, for example, reduce the weight of the biological-information detecting device 100. Further, by reducing a contact area with the wrist 200, it is also possible to, for example, reduce a local burden on skin.

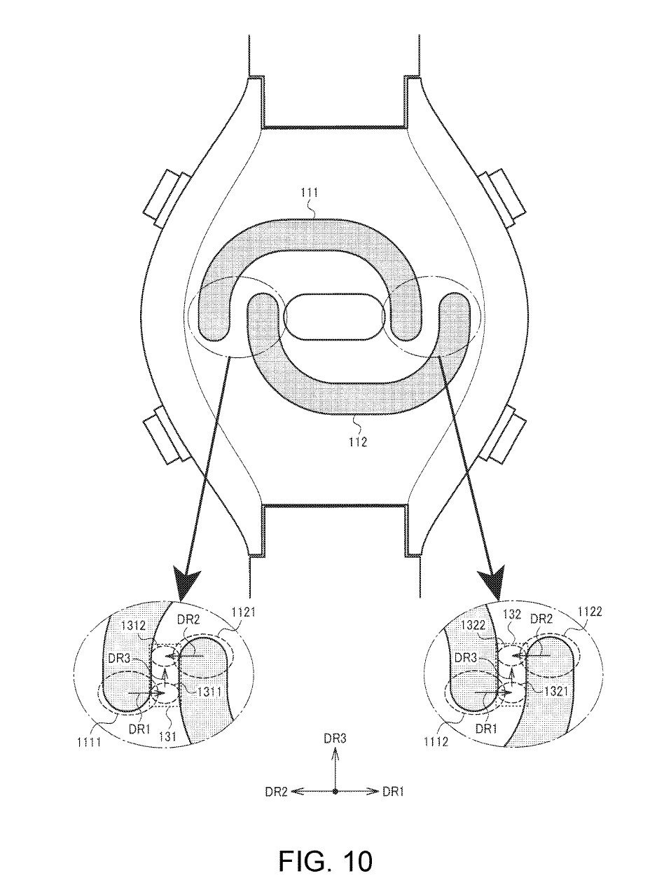

As a simpler and more effective shape of the pad section 110, a combination of two pad sections shown in FIG. 10 is also conceivable.

In an example shown in FIG. 10, the pad section 110 includes the first pad section 111 and the second pad section 112. That is, in the example shown in FIG. 10, the first pad section 111 and the second pad section 112 are disposed to mesh with each other. As enlarged and shown in the lower left of FIG. 10, the air entry path includes the first gap section 131.

The opposite direction of the first direction DR1 is represented as the second direction DR2 and a direction crossing the first direction DR1 is represented as the third direction DR3. The first gap section 131 is specifically explained.

As shown in the lower left of FIG. 10, the first gap section 131 includes a first gap section end portion 1311 and a second gap section end portion 1312. The second gap section end portion 1312 is a gap section end portion opposed to the first gap section end portion 1311. In this case, the first gap section end portion 1311 of the first gap section 131 is located on the first direction DR1 side of the first end portion 1111 of the first pad section 111. The second gap section end portion 1312 of the first gap section 131 is located on the second direction DR2 side of the first end portion 1121 of the second pad section 112 and on the third direction DR3 side of the first gap section end portion 1311.

As shown in the lower right of FIG. 10, the air entry path further includes the second gap section 132. A first gap section end portion 1321 of the second gap section 132 is located on the first direction DR1 side of the second end portion 1112 of the first pad section 111. A second gap section end portion 1322 opposed to the first gap section end portion 1321 is located on the second direction DR2 side of the second end portion 1122 of the second pad section 112 and on the third direction DR3 side of the first gap section end portion 1321 of the second gap section 132.

Consequently, it is possible to bend the air entry path to block intrusion of external light. In addition, since simple shapes are combined, it is possible to, for example, iconize a shape and obtain a shape with a high design property.

In the example shown in FIG. 10, the second pad section 112 can also be considered a pad section point-symmetrical to the first pad section 111 centering on the detection window 90. The point-symmetrical pad section means one pad section, the shape and the disposition of which substantially coincide with the shape and the disposition of the other pad section when the one pad section is rotated 180° with the center point of the detection window 90 set as a rotation axis. However, the shapes of the first pad section 111 and the second pad section 112 do not have to completely coincide with each other.

Similarly, in the example shown in FIG. 10, in the air entry path, the second gap section 132 can also be considered a gap section point-symmetrical to the first gap section 131 centering on the detection window 90. The point-symmetrical gap section means one gap section, the shape of which substantially coincides with the shape of the other gap section when the one gap section is rotated 180° with the center point of the detection window 90 set as a rotation axis. However, the shapes of the first gap section 131 and the second gap section 132 do not have to completely coincide with each other.

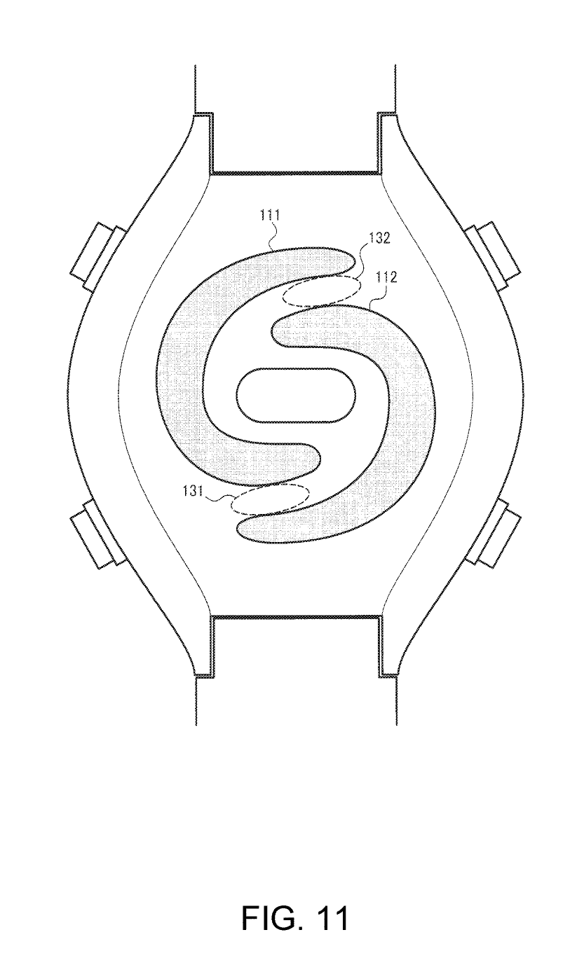

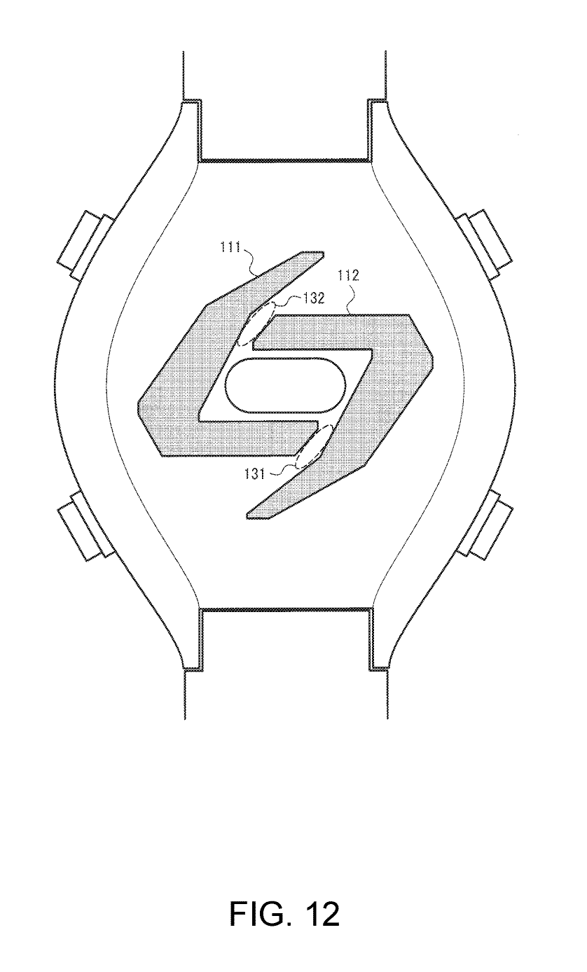

As modifications of the pad section shown in FIG. 10, modifications shown in FIGS. 11 and 12 are conceivable. However, the modifications are not limited to these modifications.

The pad section 110 is formed of a material softer than a material configuring the first surface 31 of the case section 30. For example, the case section 30 is formed of plastic, metal, or the like and the pad section 110 is formed of silicone gel, low resilience urethane foam, elastomer, or an air bag. For example, since the silicone gel has a heat radiation effect, it is possible to, for example, allow heat generated by perspiration to escape. The low resilience urethane foam is visco-elastic urethane foam having low rebound resistance and slow deformation restoration as characteristics and is excellent in a body pressure dispersion characteristic, an anti-shock property, and energy absorption. The elastomer is an industrial material having rubber elasticity. More specifically, examples of the elastomer include vulcanized rubber and thermosetting resin-based elastomer. The air bag is a bag formed of the silicone gel, the elastomer, or the like as mentioned above and filled with the air on the inside.

The pad 110 may be black. When the soft pad is colored in black, since light is absorbed, it is possible to improve the light blocking property.

Further, the pad section 110 may include small unevenness. For example, embossing may be applied to the surface of the pad section 110. The embossing is one of surface treatment and is treatment for physically forming a crease pattern (fine unevenness). By forming the fine unevenness (embossing or the like) on the surface of the pad section 110, it is possible to absorb light and improve the light blocking property.

The shape, the disposition, and the characteristics of the pad section 110 explained above are applicable not only to the pad formed of the soft material but also to a bank molded by a case of hard resin. Like the pad section 110, the bank formed in the case section 30 is also provided to prevent instruction of external light into the detection window 90. When the air entry path is provided in the bank, a pad section having a shape same as the shape of the pad section 110 shown in FIGS. 5 to 12 may be formed of hard resin.

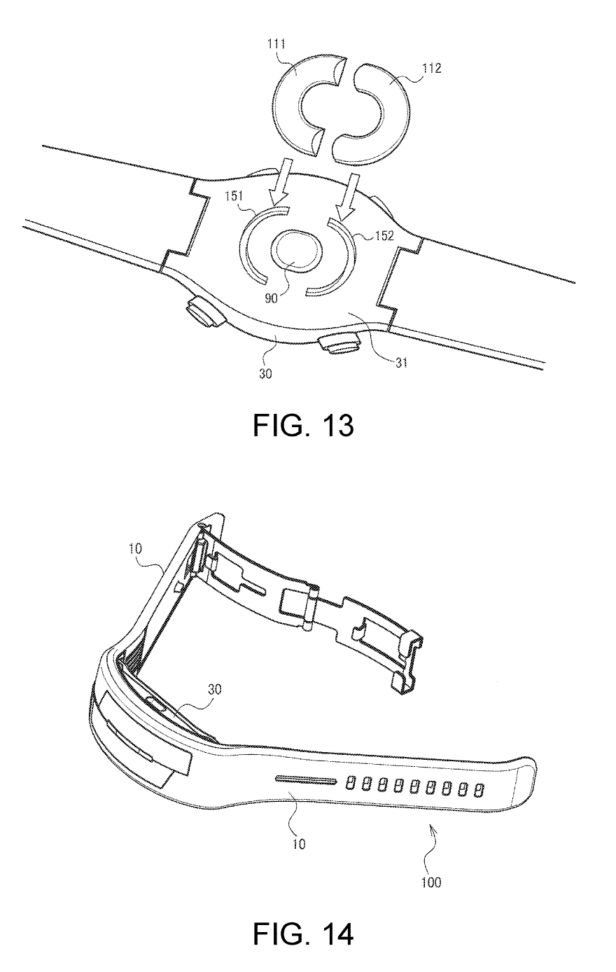

As shown in FIG. 13, the first surface 31 of the case section 30 includes a recessed section 151 (152) into which at least a part of the pad section 110 is inserted. In an example shown in FIG. 13, the first pad section 111 includes, on the rear surface, a not-shown projected section that can be fit with the recessed section 151. The first pad section 111 and the recessed section 151 are fit with each other. Similarly, the second pad section 112 includes, on the rear surface, a not-shown projected section that can be fit with the recessed section 152. The second pad section 112 and the recessed section 152 are fit with each other.

The recessed section 151 (152) is a hollow or a groove formed on the first surface 31 of the case section 30. For example, as explained above, the recessed section 151 (152) has a shape that can be fit with the projected section provided on the rear surface of the pad section.

Consequently, it is possible to, for example, prevent the pad section 110 from easily coming off the first surface 31.

This embodiment explained above is applicable to not only the biological-information detecting device 100 including the first band section 11 and the second band section 12 shown in FIG. 1 but also to the biological-information detecting device 100 including only one band section 10 shown in FIG. 14. In the biological-information detecting device 100 shown in FIG. 14, the case section 30 is provided on the rear surface of the band section 10. The pad section 110 in this embodiment is provided on a first surface (a surface on the subject side during wearing) of the case section 30.

As explained above, in the related art, deterioration in sensor accuracy due to perspiration during exercise cannot be suppressed. This is because, in the related art, only improvement of adhesion to skin is intended and an adverse effect on the optical sensor due to perspiration, which becomes obvious according to the improvement of the adhesion, is not taken into account. In this embodiment, it is possible to improve a pulse wave detection property by facilitating circulation of the air present between the detector and the wrist without encapsulating the air. Consequently, it is also possible to reduce discomfort of the wearer due to stuffiness by perspiration and accumulated sweat.

In the biological-information detecting device 100 shown in FIG. 14, the detection window 90 of the detector is sometimes not provided on the first surface of the case section 30. This embodiment is also applicable to such a biological-information detecting device 100. For example, the biological-information detecting device 100 includes the case section 30, the band section 11 (12) attached to the case section 30, and the pad section 110 provided on the first surface 31 on the subject side of the case section 30. In the pad section 110, the air entry path for the air to enter is provided in the gap between the case section 30 and the subject during wearing of the biological-information detecting device 100. In other words, the detector may be disposed on the rear surface of the case section 30 and the pad section 110 or the gap section 130 may be disposed around the detector. In this case as well, it is possible to reduce discomfort of the wearer due to stuffiness by perspiration and accumulated sweat.

This embodiment is explained in detail above. However, those skilled in the art could easily understand that many modifications are possible without substantially departing from the new matters and the effects of the invention. Therefore, all such modifications are deemed to be included in the scope of the invention. For example, terms described together with broader or synonymous different terms at least once in the specification or the drawings can be replaced with the different terms in any place in the specification or the drawings. The configurations and the operations of the biological-information detecting device are not limited to the configurations and the operations explained in the embodiments. Various modified implementations of the configurations and the operations are possible.

What is claimed is:

1. A biological-information detecting device comprising:
a case section worn on a subject, a detection window of a detector configured to detect biological information of the subject being provided on a first surface on the subject side of the case section;
a band section attached to the case section; and
a pad section provided around the detection window on the first surface of the case section, wherein
a gap for air to enter the detection window is provided in the pad section.

2. The biological-information detecting device according to claim 1, wherein
the pad section includes:
a first pad section; and
a second pad section, and
the gap includes a first gap provided between a first end portion of the first pad section and a first end portion of the second pad section opposed to the first end portion of the first pad section.

3. The biological-information detecting device according to claim 2, wherein the gap further includes a second gap provided between a second end portion of the first pad section and a second end portion of the second pad section opposed to the second end portion of the first pad section.

4. The biological-information detecting device according to claim 2, wherein the first gap is a gap extending along a longitudinal direction of the band section.

5. The biological-information detecting device according to claim 1, wherein
the pad section includes first to third pad sections, and
the gap includes:
a first gap provided between a first end portion of the first pad section and a first end portion of the second pad section opposed to the first end portion of the first pad section; and
a second gap provided between a second end portion of the second pad section and a first end portion of the third pad section opposed to the second end portion of the second pad section.

6. The biological-information detecting device according to claim 5, wherein the pad section further includes a fourth pad section, and
the gap includes a third gap provided between a second end portion of the third pad section and a first end portion of the fourth pad section opposed to the second end portion of the third pad section.

7. The biological-information detecting device according to claim 6, wherein the gap includes a fourth gap provided between a second end portion of the fourth pad section and a second end portion of the first pad section opposed to the second end portion of the fourth pad section.

8. The biological-information detecting device according to claim 1, wherein the gap has a bent shape or a curved shape.

9. The biological-information detecting device according to claim 1, wherein
the pad section includes:
a first pad section provided around the detection window; and
a second pad section provided around the first pad section,
in the first pad section, a first gap is provided on a first direction side of the detection window, and
in the second pad section, when a direction crossing the first direction is represented as a second direction, a second gap is provided in the second direction side of the detection window.

10. The biological-information detecting device according to claim 1, wherein
the pad section includes:
a first pad section; and
a second pad section,
the gap includes a first gap, and
when an opposite direction of the first direction is represented as a second direction and a direction crossing the first direction is represented as a third direction, a first gap end portion of the first gap is located on the first direction side of a first end of the first pad section and a second gap end portion of the first gap is located on the second direction side of a first end portion of the second pad section and on the third direction side of the first gap end portion.

11. The biological-information detecting device according to claim 1, wherein
the pad section includes:
a first pad section; and
a second pad section, and
the second pad section is a pad section point-symmetrical to the first pad section centering on the detection window.

12. The biological-information detecting device according to claim 1, wherein
the gap includes:
a first gap; and
a second gap, and
the second gap is a gap point-symmetrical to the first gap centering on the detection window.

13. The biological-information detecting device according to claim 1, wherein the pad section is formed of a material softer than a material forming the first surface of the case section.

14. The biological-information detecting device according to claim 1, wherein the first surface of the case section includes a recessed section into which at least a part of the pad section is inserted.
